(19) United States
(12) Patent Application Publication
(10) Pub. No.: US 2025/0033035 A1
(43) Pub. Date: Jan. 30, 2025

(54) BIOPARTICLE CONTACTLESS PROCESSING APPARATUS

(71) Applicant: CYTOAURORA BIOTECHNOLOGIES, INC., ZHUBEI CITY (TW)

(72) Inventors: Chung-Er Huang, ZHUBEI CITY (TW); Hsin-Cheng Ho, ZHUBEI CITY (TW)

(21) Appl. No.: 18/780,487

(22) Filed: Jul. 23, 2024

Related U.S. Application Data
(60) Provisional application No. 63/528,896, filed on Jul. 25, 2023.

(51) Int. Cl. B01L 3/00

(52) U.S. Cl. CPC B01L 3/502; B01L 2300/0627; B01L 2300/0645; B01L 2300/088

(57) ABSTRACT

A bioparticle contactless processing apparatus includes an accommodating device and a triggering device. The accommodating device includes a light sensing structure, a mating structure spaced apart from the light sensing structure, and a frame arranged between the light sensing structure and the mating structure. The frame has two working segments each having a first opening and a second opening that is smaller than the first opening, and the first opening and the second opening are respectively arranged on two opposite ends of the two working segments. The triggering device is arranged corresponding to the frame. When at least one bioparticle is transferred into the frame by passing through the first opening and has a size larger than the second opening, the triggering device is configured to trigger an outer cell layer of the at least one bioparticle to have a predetermined permeability higher than an original permeability thereof.

### CROSS-REFERENCE TO RELATED PATENT APPLICATION

This application claims the benefit of priority to the U.S. Provisional Patent Application Ser. No. 63/528,896, filed on Jul. 25, 2023, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a bioparticle processing apparatus, and more particularly to a bioparticle contactless processing apparatus.

### BACKGROUND OF THE DISCLOSURE

A conventional bioparticle processing device can be operated in a light driving manner for moving a bioparticle. However, the bioparticle is difficult to be precisely controlled by the conventional bioparticle processing device to be located at a fixed point for relevant processing.

### SUMMARY OF THE DISCLOSURE

In response to the above-referenced technical inadequacies, the present disclosure provides a bioparticle contactless processing apparatus for effectively improving on the issues associated with conventional bioparticle processing devices.

In order to solve the above-mentioned problems, one of the technical aspects adopted by the present disclosure is to provide a bioparticle contactless processing apparatus, which includes an accommodating device, a light capturing device, and a triggering device. The accommodating device is provided for receiving a liquid specimen that has a plurality of bioparticles and a transfection substance. The accommodating device includes a light sensing structure, a mating structure, and a frame. The light sensing structure includes a first substrate, a first electrode layer formed on the first substrate, and a photoelectric layer that is formed on the first substrate. The mating structure is spaced apart from the light sensing structure. At least one of the mating structure and the light sensing structure is transparent, and the mating structure includes a second substrate and a second electrode layer that is formed on the second substrate and that faces toward the light sensing structure. The frame is arranged between the light sensing structure and the mating structure. The frame has two working segments facing toward each other and has two opposite ends that respectively define a first opening and a second opening being smaller than the first opening. At least one of the bioparticles is defined as a target bioparticle having a particle size being greater than the second opening, and an outer cell layer of the target bioparticle has an original permeability. The light capturing device faces toward the accommodating device. The light capturing device is configured to drive the light sensing structure to form a first dielectrophoresis (DEP) pattern that is capable of moving the target bioparticle to a location between the two working segments by passing through the first opening. The triggering device is disposed corresponding in position to the frame. The triggering device is configured to trigger the outer cell layer of the target bioparticle located between the two working segments, whereby the outer cell layer has a predetermined permeability being greater than the original permeability. When the target bioparticle is captured to be located between the two working segments, the light capturing device is configured to drive the light sensing structure to form a second DEP pattern that is capable of moving the transfection substance to a location between the two working segments by passing through the second opening, thereby performing a transfection process.

In order to solve the above-mentioned problems, another one of the technical aspects adopted by the present disclosure is to provide a bioparticle contactless processing apparatus, which includes an accommodating device, a light capturing device, and a triggering device. The accommodating device is provided for receiving a liquid specimen that has a plurality of bioparticles. The accommodating device includes a light sensing structure, a mating structure, and a frame. The light sensing structure includes a first substrate, a first electrode layer formed on the first substrate, and a photoelectric layer that is formed on the first substrate. The mating structure is spaced apart from the light sensing structure. At least one of the mating structure and the light sensing structure is transparent, and the mating structure includes a second substrate and a second electrode layer that is formed on the second substrate and that faces toward the light sensing structure. The frame is arranged between the light sensing structure and the mating structure. The frame has two working segments facing toward each other and has two opposite ends that respectively define a first opening and a second opening being smaller than the first opening. At least one of the bioparticles is defined as a target bioparticle having a particle size being greater than the second opening, and an outer cell layer of the target bioparticle has an original permeability. The light capturing device faces toward the accommodating device. The light capturing device is configured to drive the light sensing structure to form a first dielectrophoresis (DEP) pattern that is capable of moving the target bioparticle to a location between the two working segments by passing through the first opening. The triggering device is disposed corresponding in position to the frame. The triggering device is configured to trigger the outer cell layer of the target bioparticle located between the two working segments, whereby the outer cell layer has a predetermined permeability being greater than the original permeability for enabling the target bioparticle to generate an exosome passing through the outer cell layer. When the target bioparticle is captured to be located between the two working segments and the exosome is generated from the target bioparticle, the light capturing device is configured to drive the light sensing structure to form a second DEP pattern that is capable of moving the exosome to a location outside of the two working segments by passing through the second opening, thereby performing a purification process.

In order to solve the above-mentioned problems, yet another one of the technical aspects adopted by the present disclosure is to provide a bioparticle contactless processing apparatus, which includes an accommodating device and a triggering device. The accommodating device is provided for receiving a liquid specimen that has a plurality of bioparticles. The accommodating device includes a light sensing structure, a mating structure, and a frame. The light sensing structure includes a first substrate, a first electrode layer formed on the first substrate, and a photoelectric layer that is formed on the first substrate. The mating structure is spaced apart from the light sensing structure. At least one of the mating structure and the light sensing structure is transparent, and the mating structure includes a second substrate and a second electrode layer that is formed on the second substrate and that faces toward the light sensing structure. The frame is arranged between the light sensing structure and the mating structure. The frame has two working segments facing toward each other and has two opposite ends that respectively define a first opening and a second opening being smaller than the first opening. At least one of the bioparticles is defined as a target bioparticle having a particle size being greater than the second opening, and an outer cell layer of the target bioparticle has an original permeability. The triggering device is disposed corresponding in position to the frame. When the target bioparticle is located between the two working segments by passing through the first opening, the triggering device is configured to trigger the outer cell layer of the target bioparticle, whereby the outer cell layer has a predetermined permeability being greater than the original permeability.

Therefore, the frame and the triggering device in the bioparticle contactless processing apparatus provided by the present disclosure are cooperated with each other and are in cooperation with the light sensing structure of the accommodating device, such that the frame can be used to position or hold the target bioparticle, and the triggering device can be used to increase the permeability of the outer cell layer of the target bioparticle according to practical requirements, thereby enabling a relevant process (e.g., the transfection process or the purification process) to be precisely performed on the target bioparticle.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a bioparticle contactless processing apparatus according to a first embodiment of the present disclosure;

FIG. 2 is a schematic planar cross-sectional view of the bioparticle contactless processing apparatus according to the first embodiment of the present disclosure;

FIG. 3 is a schematic perspective cross-sectional view of the bioparticle contactless processing apparatus according to the first embodiment of the present disclosure;

FIG. 4 is a schematic cross-sectional view showing the bioparticle contactless processing apparatus of FIG. 2 in another configuration according to the first embodiment of the present disclosure;

FIG. 5 is a schematic cross-sectional view showing a follow-up action of the bioparticle contactless processing apparatus of FIG. 2;

FIG. 6 is a schematic cross-sectional view showing a follow-up action of the bioparticle contactless processing apparatus of FIG. 6;

FIG. 7 is a schematic cross-sectional view of the bioparticle contactless processing apparatus according to a second embodiment of the present disclosure;

FIG. 8 is a schematic cross-sectional view of the bioparticle contactless processing apparatus according to a third embodiment of the present disclosure;

FIG. 9 is a schematic cross-sectional view showing a follow-up action of the bioparticle contactless processing apparatus of FIG. 8; and

FIG. 10 is a schematic cross-sectional view showing a follow-up action of the bioparticle contactless processing apparatus of FIG. 9.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

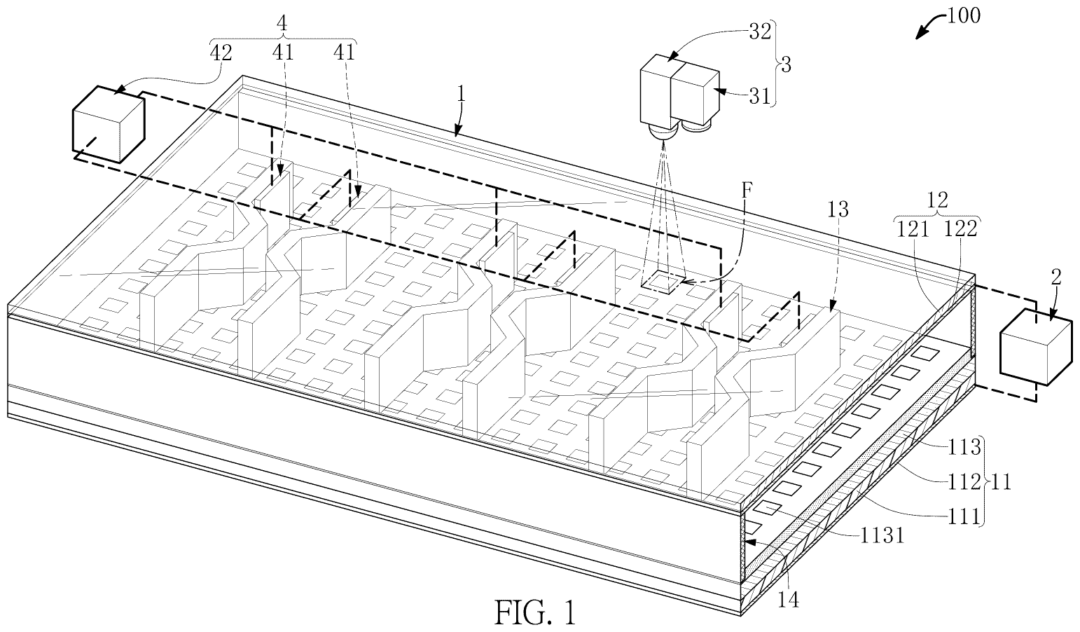

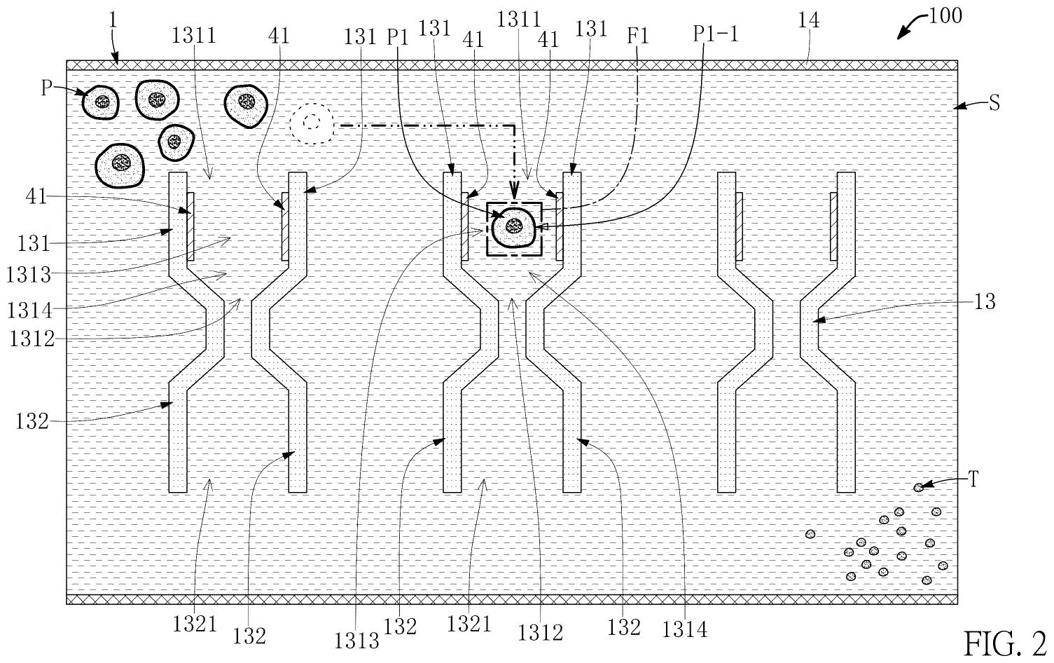

Referring to FIG. 1 to FIG. 6, a first embodiment of the present disclosure is provided. As shown in FIG. 1 to FIG. 3, the present embodiment provides a bioparticle contactless processing apparatus 100, which includes an accommodating device 1, an alternating current (AC) power device 2 electrically coupled to the accommodating device 1, a light capturing device 3 facing toward the accommodating device 1, and a triggering device 4 that is at least partially disposed in the accommodating device 1, but the present disclosure is not limited thereto. For example, in other embodiments of the present disclosure not shown in the drawings, the bioparticle contactless processing apparatus 100 can be provided without at least one of the light capturing device 3 and the triggering device 4 according to practical requirements. In other words, the accommodating device 1 and the triggering device 4 can be in cooperation with each other for being independently used (e.g., sold) or can be used in cooperation with other devices.

The accommodating device 1 in the present embodiment is formed at a chip-scale and is a rectangular structure, and the accommodating device 1 is provided for receiving a liquid specimen S that has a plurality of bioparticles P and a transfection substance T therein, but the present disclosure is not limited thereto. For example, a quantity of the bioparticles P in the liquid specimen S can be adjusted (e.g., at least one) according to practical requirements.

It should be noted that the liquid specimen S can be a body fluid from an animal (e.g., blood, lymph, saliva, or urine), and the bioparticle P can be a specific type of cell or cell clusters, such as circulating tumor cells (CTCs), fetal nucleated red blood cells (FNRBCs), or bacteria, and the transfection substance T has genetic material and can be at least one of RNA, DNA, exosome, liposome, and virus, but the present disclosure is not limited thereto. For example, in other embodiments of the present disclosure not shown in the drawings, the liquid specimen S can also be obtained from plants.

The accommodating device 1 includes a light sensing structure 11, a mating structure 12 spaced apart from the light sensing structure 11, a plurality of frames 13 arranged (or sandwiched) between the light sensing structure 11 and the mating structure 12, and a bonding layer 14 that connects the light sensing structure 11 and the mating structure 12. At least one of the mating structure 12 and the light sensing structure 11 is transparent. The light sensing structure 11 and the mating structure 12 in the present embodiment are flat structures parallel to each other and are spaced apart from each other by a distance that is greater than a particle size of any one of the bioparticles P, but the present disclosure is not limited thereto.

Specifically, the light sensing structure 11 includes a first substrate 111, a first electrode layer 112 formed on the first substrate 111, and a photoelectric layer 113 formed on the first substrate 111. In the present embodiment, the first electrode layer 112 is formed on a bottom side of the first substrate 111, the photoelectric layer 113 is formed on a top side of the first substrate 111, and the first electrode layer 112 has a plurality of transistors being in a matrix arrangement, but the present disclosure is not limited thereto.

The mating structure 12 includes a second substrate 121 and a second electrode layer 122 that is formed on the second substrate 121 and that faces toward the light sensing structure 11 (e.g., the photoelectric layer 113). In the present embodiment, the AC power device 2 is electrically coupled to the first electrode layer 112 of the light sensing structure 11 and the second electrode layer 122 of the mating structure 12, such that the light sensing structure 11 can be irradiated by light emitted from the light capturing device 3 for forming a dielectrophoresis (DEP) pattern F that is capable of moving at least one of the bioparticles P or the transfection substance T in the liquid specimen S.

For example, the light capturing device 3 includes a camera 31 and a light source 32 that is in cooperation with the camera 31. The light sensing structure 11 can be irradiated by light emitted from the light source 32 for forming the DEP pattern F.

The frames 13 are sandwiched between the photoelectric layer 113 of the light sensing structure 11 and the second electrode layer 122 of the mating structure 12. Moreover, as the frames 13 in the present embodiment are of substantially a same structure, the following description discloses the structure of just one of the frames 13 for the sake of brevity, but the present disclosure is not limited thereto. For example, in other embodiments of the present disclosure not shown in the drawings, the frames 13 can be of different structures.

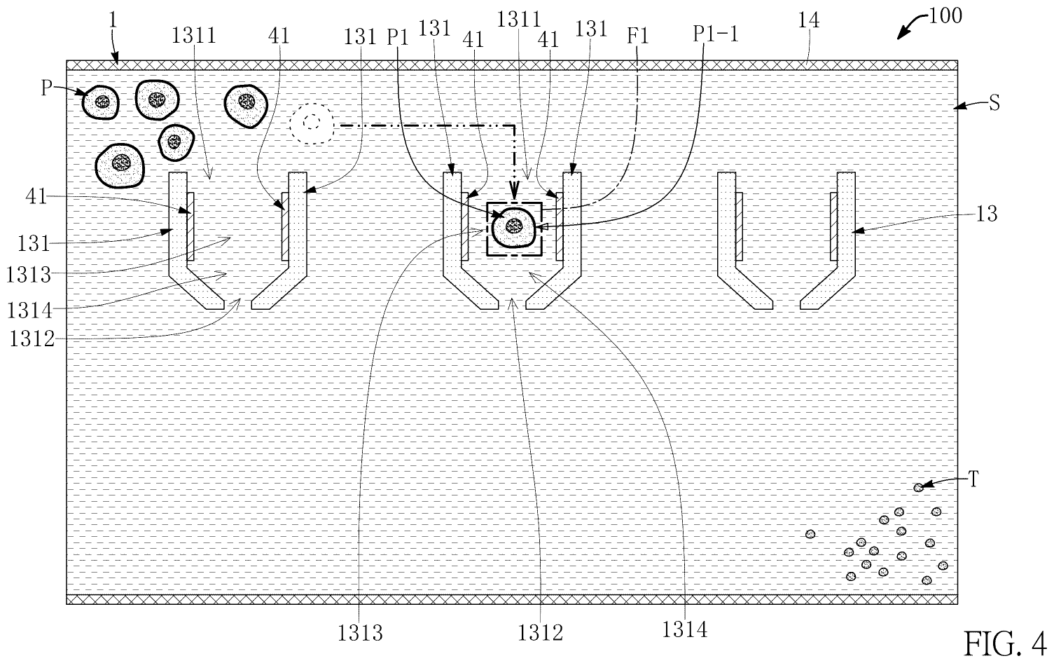

In the present embodiment, the frame 13 is a mirror symmetrical structure, and the frame 13 includes two working segments 131 and two channel segments 132 that respectively extend from the two working segments 131. The two working segments 131 face toward each other and are spaced apart from each other, and the channel segments 132 face toward each other and are spaced apart from each other, but the present disclosure is not limited thereto. For example, as shown in FIG. 4, the two channel segments 132 can be omitted according to practical requirements.

Specifically, two opposite ends (e.g., a top end and a bottom end) of the two working segments 131 respectively have or define a first opening 1311 and a second opening 1312 that is smaller than the first opening 1311. In other words, a space between the two working segments 131 can be in spatial communication with an external space through the first opening 1311 and/or the second opening 1312. The two channel segments 132 are respectively connected to one of the two opposite ends of the two working segments 131 that defines the second opening 1312, and the two channel segments 132 define a third opening 132 that is arranged away from the second opening 1312 and that is greater than the second opening 1312. In the present embodiment, a size of the third opening 132 is substantially equal to a size of the first opening 1311, but the present disclosure is not limited thereto.

Specifically, the space between the two working segments 131 in the present embodiment has a receiving region 1313 and a neck region 1314 that is in spatial communication with the receiving region 1313. The first opening 1311 is arranged or defined at one end of the receiving region 1313, and the neck region 1314 defines the second opening 1312 arranged away from the first opening 1311. Moreover, a width of the receiving region 1313 is substantially equal to the first opening 1311, the neck region 1314 is arranged at another end of the receiving region 1313, and the neck region 1314 is tapered in a direction away from the receiving region 1313 so as to define the second opening 1312, but the present disclosure is not limited thereto.

It should be noted that at least one of the bioparticles P is defined as a target bioparticle P1 having a particle size that is greater than the second opening 1312 and that is less than the first opening 1311. An outer cell layer P1-1 (e.g., a membrane or a wall) of the target bioparticle P1 has an original permeability. A size of the transfection substance T is less than the second opening 1312.

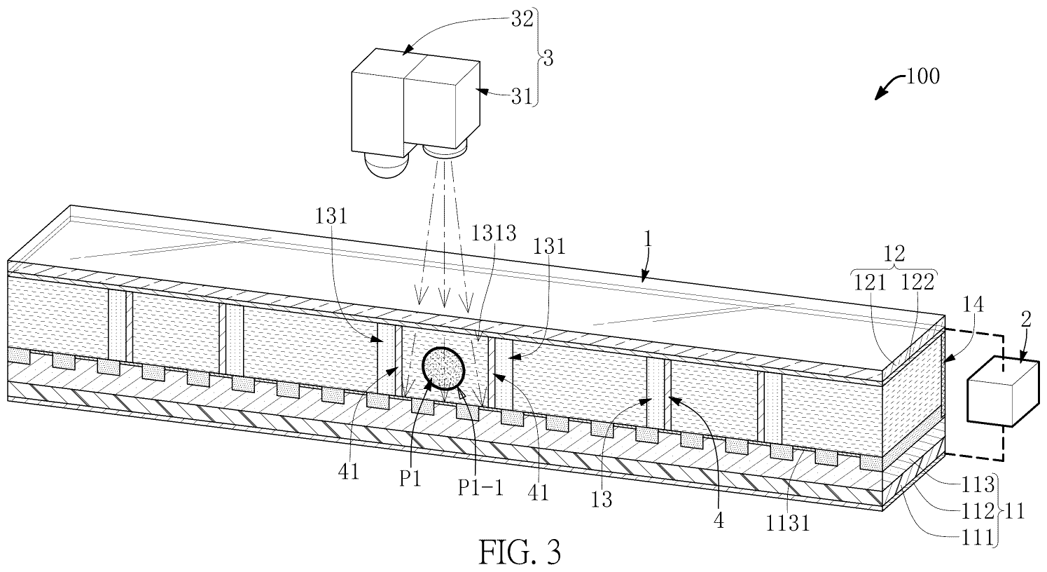

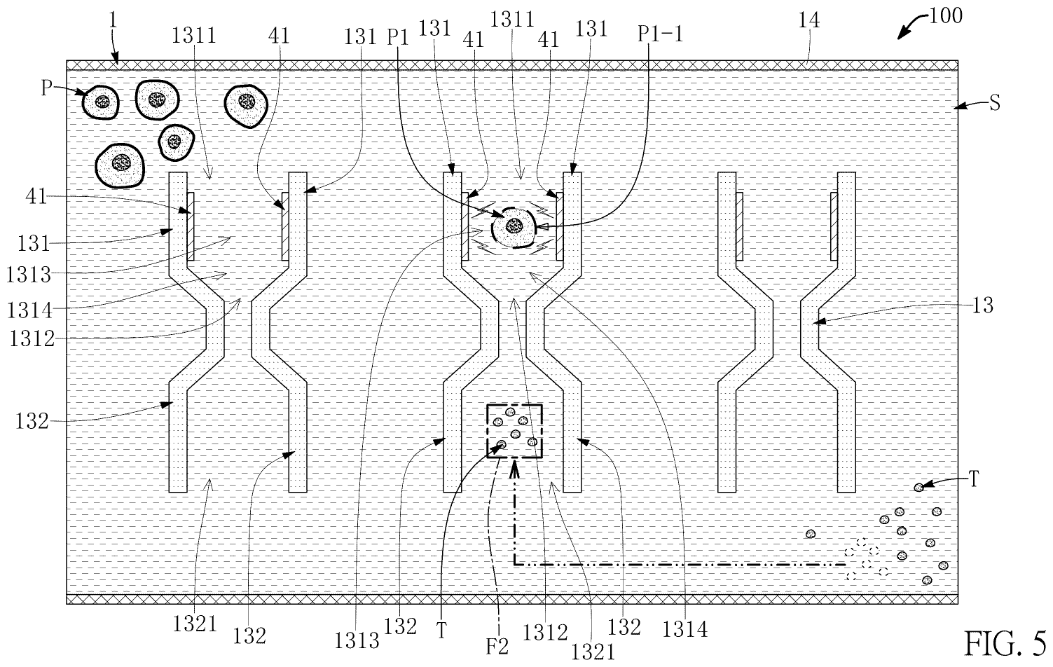

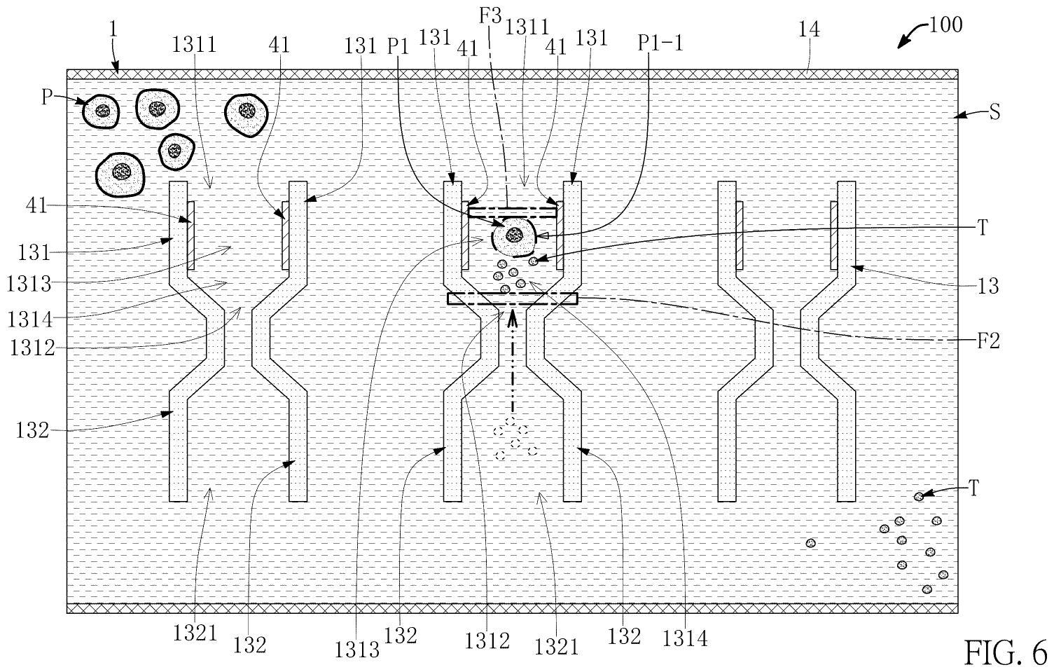

As shown in FIG. 3, FIG. 5, and FIG. 6, the light capturing device 3 is configured to drive the light sensing structure 11 to form a first DEP pattern F1 that is capable of moving the target bioparticle P1 to a location between the two working segments 131 by passing through the first opening 1311. In the present embodiment, light emitted from the light capturing device 3 irradiates onto the light sensing structure 11 to form the first DEP pattern F1 having an enclosed shape surrounding the target bioparticle P1, such that the light capturing device 3 can control a movement of the first DEP pattern F1 for moving the target bioparticle P1 into the receiving space 1313 by passing through the first opening 1311.

Specifically, when the target bioparticle P1 is captured to be located between the two working segments 131, the light capturing device 3 is configured to drive the light sensing structure 11 to form a second DEP pattern F2 that is capable of moving the transfection substance T to a location between the two working segments 131 by passing through the second opening 1312, thereby performing a transfection process. Accordingly, when the transfection substance T includes virus, the target bioparticle P1 in the transfection process is captured to be located between the two working segments 131 and has the original permeability.

It should be noted that when the target bioparticle P is captured to be located between the two working segments 131, the light capturing device 3 is configured to drive the light sensing structure 11 to form a third DEP pattern F3 that is capable of maintaining the target bioparticle P1 to a location between the two working segments 131, thereby facilitating the implementation of the transfection process. Moreover, the shapes of the second DEP pattern F2 and the third DEP pattern F3 can be adjusted or changed according to practical requirements.

In addition, the position of the target bioparticle P1 in the present embodiment is limited by the first DEP pattern F3, but the present disclosure is not limited thereto. In other embodiments of the present disclosure not shown in the drawings, the target bioparticle P1 can be limited at a specific position through a pressure control of the liquid specimen S.

The triggering device 4 is disposed corresponding in position to the frame 13. The triggering device 4 is configured to trigger the outer cell layer P1-1 of the target bioparticle P1 located between the two working segments 131, whereby the outer cell layer P1-1 has a predetermined permeability being greater than the original permeability. The triggering device 4 can transiently increase the permeability of the outer cell layer P1-1 in an electrical manner or an optical manner (e.g., an electroporation manner, a laserfection manner, or an opoinjection manner). In order to clearly describe the present embodiment, the triggering device 4 described in the following description is operated in the electroporation manner, but the present disclosure is not limited thereto.

Specifically, the triggering device 4 includes two electrode pads 41 and a power source 42 that is electrically coupled to the two electrode pads 41. In the present embodiment, the power source 42 is a direct current (DC) power source, and the two electrode pads 41 are a positive electrode and a negative electrode (e.g., the two electrode pads 41 are located in the receiving region 1313 and are respectively disposed on inner walls of the two working segments 131), but the present disclosure is not limited thereto. For example, in other embodiments of the present disclosure not shown in the drawings, the power source 42 can be an alternating current (AC) power source according to practical requirements.

When the target bioparticle P1 is captured to be located in the receiving region 1313, the power source 42 is configured to drive the two electrode pads 41 to apply an electric field (e.g., a short high-voltage pulses) to the target bioparticle P1, whereby an electroporation is formed in the target bioparticle P1 for enabling the outer cell layer P1-1 to have the predetermined permeability.

Accordingly, when the transfection substance T includes at least one of RNA, DNA, exosome, and liposome, the target bioparticle P1 in the transfection process is captured to be located between the two working segments 131 and has the predetermined permeability through the triggering device 4.

In summary, the frame 13 and the triggering device 4 in the bioparticle contactless processing apparatus 100 provided by the present embodiment are cooperated with each other and are in cooperation with the light sensing structure 11 of the accommodating device 1, such that the frame 13 can be used to position or hold the target bioparticle P1, and the triggering device 4 can be used to increase the permeability of the outer cell layer P1-1 of the target bioparticle P1 according to practical requirements, thereby precisely performing any process on the target bioparticle P1.

### Second Embodiment

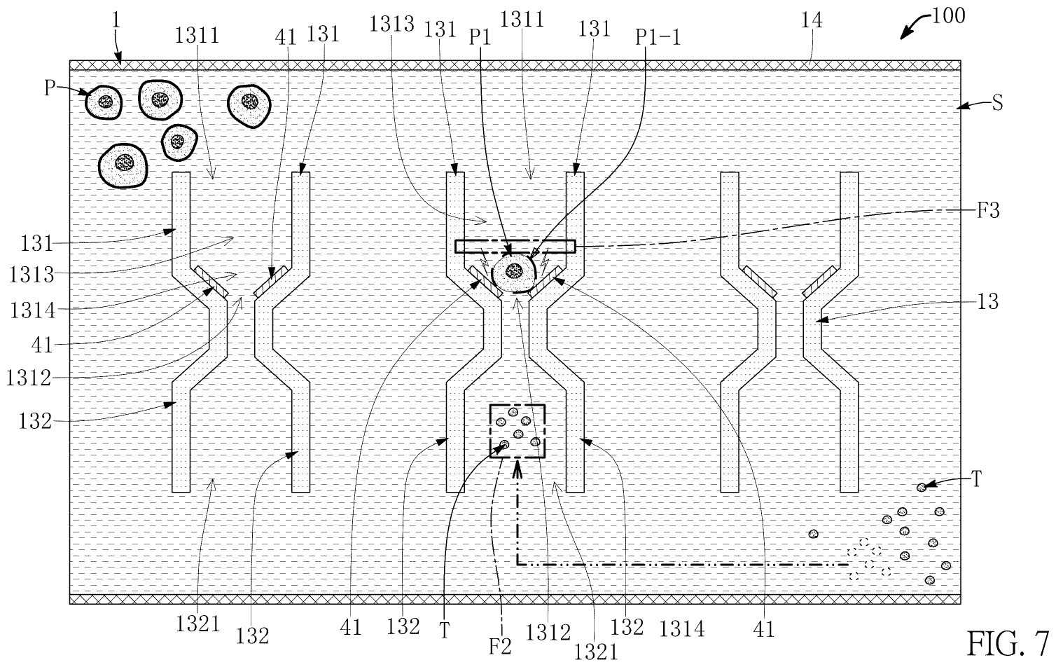

Referring to FIG. 7, a second embodiment of the present disclosure, which is similar to the first embodiment of the present disclosure, is provided. For the sake of brevity, descriptions of the same components in the first and second embodiments of the present disclosure will be omitted herein, and the following description only discloses different features between the first and second embodiments (e.g., structures of the triggering device 4).

In the present embodiment, the two electrode pads 41 are located in the neck region 1314 and are respectively disposed on inner walls of the two working segments 131. When the target bioparticle P1 is captured to be located in the neck region 1314, the power source 42 is configured to drive the two electrode pads 41 to apply an electric field (e.g., a short high-voltage pulses) to the target bioparticle P1, whereby an electroporation is formed in the target bioparticle P1 for enabling the outer cell layer P1-1 to have the predetermined permeability.

Accordingly, the light capturing device 3 is configured to drive the light sensing structure 11 to form the third DEP pattern F3 that is capable of maintaining or holding the target bioparticle P1 to a location in the neck region 1314, and a portion of the outer cell layer P1-1 adjacent to the second opening 1312 can be transiently opened through the electroporation, so that the light capturing device 3 is configured to drive the light sensing structure 11 to form the second DEP pattern F2 that is capable of moving the transfection substance T to a location in the neck region 1314 by passing through the second opening 1312, thereby facilitating the implementation of the transfection process of the target bioparticle P1 of the transfection substance T.

### Third Embodiment

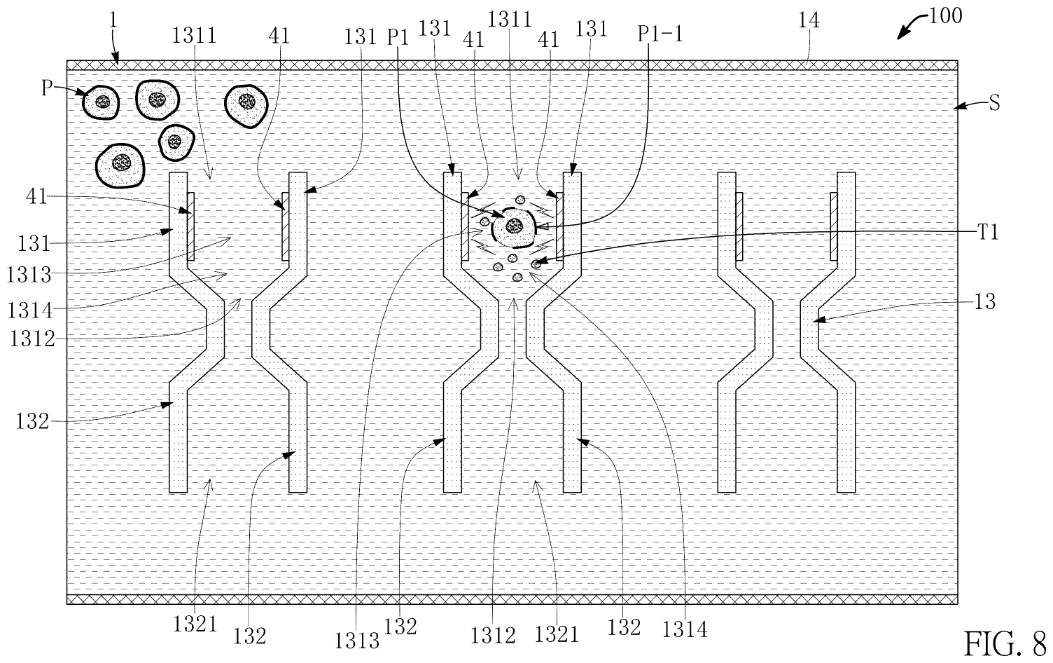

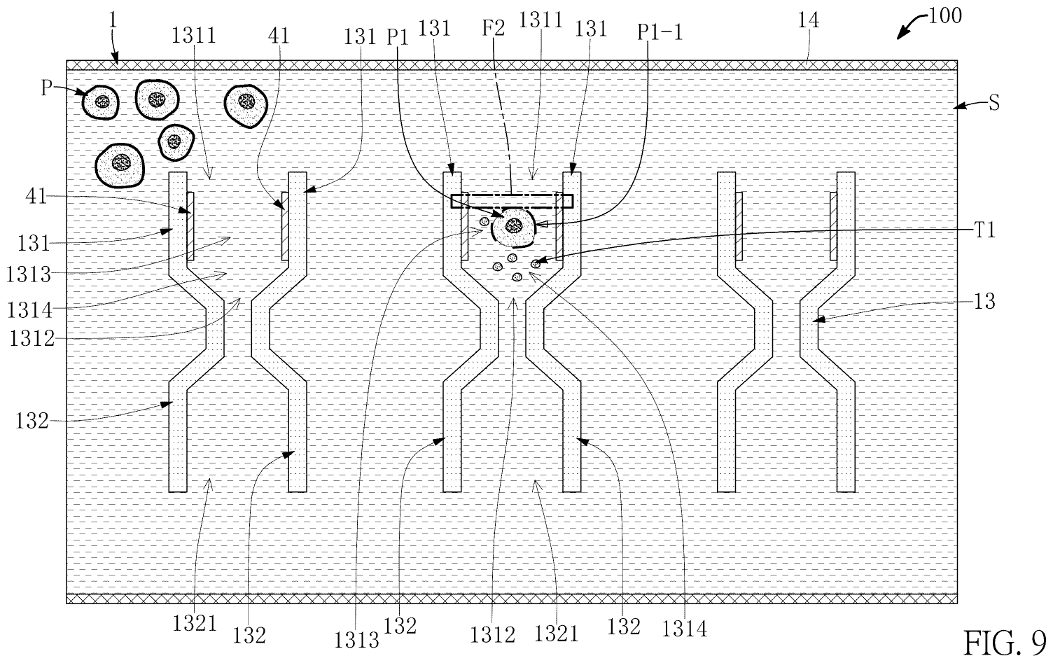

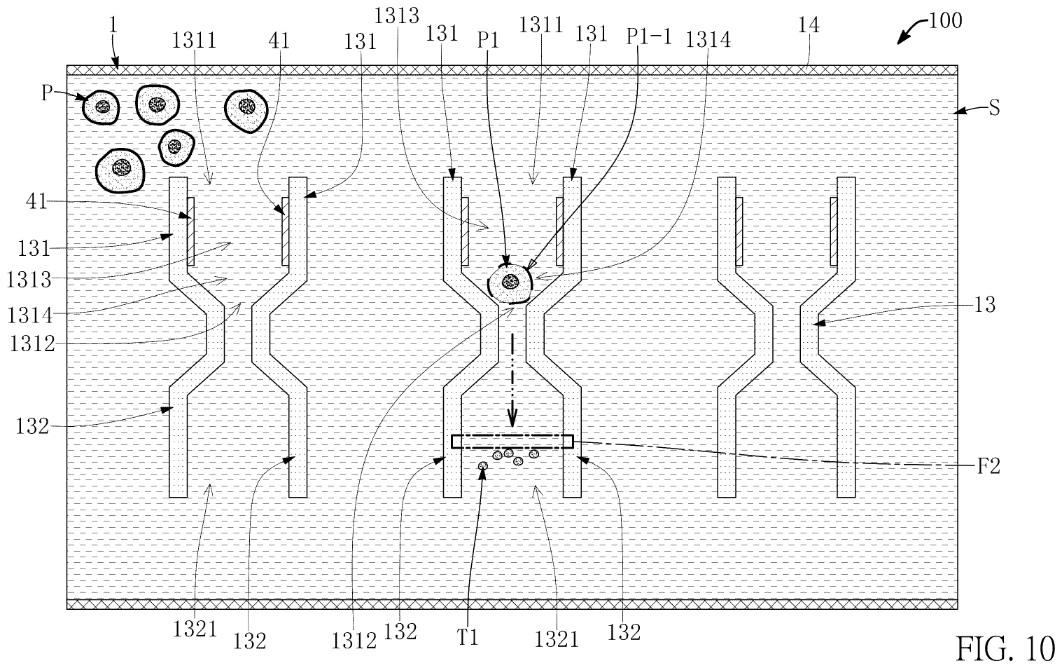

Referring to FIG. 8 to FIG. 10, a third embodiment of the present disclosure, which is similar to the first and second embodiments of the present disclosure, is provided. For the sake of brevity, descriptions of the same components in the first to third embodiments of the present disclosure will be omitted herein, and the following description only discloses different features among the first to third embodiments (e.g., an operation of the triggering device 4).

In the present embodiment, the triggering device 4 is configured to trigger the outer cell layer P1-1 of the target bioparticle P1 located between the two working segments 131, whereby the outer cell layer P1-1 has a predetermined permeability being greater than the original permeability for enabling the target bioparticle P1 to generate an exosome T1 passing through the outer cell layer P1-1.

When the target bioparticle P1 is captured to be located between the two working segments 131 and the exosome T1 is generated from the target bioparticle P1, the light capturing device 3 is configured to drive the light sensing structure 11 to form a second DEP pattern F2 that is capable of moving the exosome T1 to a location outside of the two working segments 131 by passing through the second opening 1312, thereby performing a purification process.

### Beneficial Effects of the Embodiments

In conclusion, the frame and the triggering device in the bioparticle contactless processing apparatus provided by the present disclosure are cooperated with each other and are in cooperation with the light sensing structure of the accommodating device, such that the frame can be used to position or hold the target bioparticle, and the triggering device can be used to increase the permeability of the outer cell layer of the target bioparticle according to practical requirements, thereby precisely performing any process (e.g., the transfection process or the purification process) to the target bioparticle.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A bioparticle contactless processing apparatus, comprising:
an accommodating device for receiving a liquid specimen that has a plurality of bioparticles and a transfection substance, wherein the accommodating device includes:
a light sensing structure including a first substrate, a first electrode layer formed on the first substrate, and a photoelectric layer that is formed on the first substrate;
a mating structure spaced apart from the light sensing structure, wherein at least one of the mating structure and the light sensing structure is transparent, and the mating structure includes a second substrate and a second electrode layer that is formed on the second substrate and that faces toward the light sensing structure; and
a frame arranged between the light sensing structure and the mating structure, wherein the frame has two working segments facing toward each other and having two opposite ends that respectively define a first opening and a second opening being smaller than the first opening, and wherein at least one of the bioparticles is defined as a target bioparticle having a particle size being greater than the second opening, and an outer cell layer of the target bioparticle has an original permeability;
a light capturing device facing toward the accommodating device, wherein the light capturing device is configured to drive the light sensing structure to form a first dielectrophoresis (DEP) pattern that is capable of moving the target bioparticle to a location between the two working segments by passing through the first opening; and
a triggering device disposed corresponding in position to the frame, wherein the triggering device is configured to trigger the outer cell layer of the target bioparticle located between the two working segments, whereby the outer cell layer has a predetermined permeability being greater than the original permeability;
wherein, when the target bioparticle is captured to be located between the two working segments, the light capturing device is configured to drive the light sensing structure to form a second DEP pattern that is capable of moving the transfection substance to a location between the two working segments by passing through the second opening, thereby performing a transfection process.

2. The bioparticle contactless processing apparatus according to claim 1, wherein a space between the two working segments has:
a receiving region defining the first opening arranged at one end thereof; and
a neck region arranged at another end of the receiving region, wherein the neck region is tapered in a direction away from the receiving region, and the neck region defines the second opening arranged away from the first opening.

3. The bioparticle contactless processing apparatus according to claim 2, wherein the triggering device includes:
two electrode pads located in the receiving region and respectively disposed on inner walls of the two working segments; and
a power source electrically coupled to the two electrode pads, wherein, when the target bioparticle is captured to be located in the receiving region, the power source is configured to drive the two electrode pads to apply an electric field to the target bioparticle, whereby an electroporation is formed in the target bioparticle for enabling the outer cell layer to have the predetermined permeability.

4. The bioparticle contactless processing apparatus according to claim 2, wherein the triggering device includes:
two electrode pads located in the neck region and respectively disposed on inner walls of the two working segments; and
a power source electrically coupled to the two electrode pads, wherein, when the target bioparticle is captured to be located in the neck region, the power source is configured to drive the two electrode pads to apply an electric field to the target bioparticle, whereby an electroporation is formed in the target bioparticle for enabling the outer cell layer to have the predetermined permeability.

5. The bioparticle contactless processing apparatus according to claim 1, wherein the frame includes two channel segments respectively connected to one of the two opposite ends of the two working segments that defines the second opening, and wherein the two channel segments define a third opening that is arranged away from the second opening and that is greater than the second opening.

6. The bioparticle contactless processing apparatus according to claim 1, wherein, when the target bioparticle is captured to be located between the two working segments, the light capturing device is configured to drive the light sensing structure to form a third DEP pattern that is capable of maintaining the target bioparticle at a location between the two working segments.

7. The bioparticle contactless processing apparatus according to claim 1, wherein the transfection substance includes at least one of RNA, DNA, exosome, liposome, and virus.

8. The bioparticle contactless processing apparatus according to claim 1, wherein the transfection substance includes at least one of RNA, DNA, exosome, and liposome, and wherein, in the transfection process, the target bioparticle is captured to be located between the two working segments and has the predetermined permeability.

9. The bioparticle contactless processing apparatus according to claim 1, wherein the transfection substance includes virus, and wherein, in the transfection process, the target bioparticle is captured to be located between the two working segments and has the original permeability.

10. A bioparticle contactless processing apparatus, comprising:
an accommodating device for receiving a liquid specimen that has a plurality of bioparticles, wherein the accommodating device includes:
a light sensing structure including a first substrate, a first electrode layer formed on the first substrate, and a photoelectric layer that is formed on the first substrate;
a mating structure spaced apart from the light sensing structure, wherein at least one of the mating structure and the light sensing structure is transparent, and the mating structure includes a second substrate and a second electrode layer that is formed on the second substrate and that faces toward the light sensing structure; and
a frame arranged between the light sensing structure and the mating structure, wherein the frame has two working segments facing toward each other and having two opposite ends that respectively define a first opening and a second opening being smaller than the first opening, and wherein at least one of the bioparticles is defined as a target bioparticle having a particle size being greater than the second opening, and an outer cell layer of the target bioparticle has an original permeability;
a light capturing device facing toward the accommodating device, wherein the light capturing device is configured to drive the light sensing structure to form a first dielectrophoresis (DEP) pattern that is capable of moving the target bioparticle to a location between the two working segments by passing through the first opening; and
a triggering device disposed corresponding in position to the frame, wherein the triggering device is configured to trigger the outer cell layer of the target bioparticle located between the two working segments, whereby the outer cell layer has a predetermined permeability being greater than the original permeability for enabling the target bioparticle to generate an exosome passing through the outer cell layer;
wherein, when the target bioparticle is captured to be located between the two working segments and the exosome is generated from the target bioparticle, the light capturing device is configured to drive the light sensing structure to form a second DEP pattern that is capable of moving the exosome to a location outside of the two working segments by passing through the second opening, thereby performing a purification process.

11. The bioparticle contactless processing apparatus according to claim 10, wherein a space between the two working segments has:
a receiving region defining the first opening arranged at one end thereof; and
a neck region arranged at another end of the receiving region, wherein the neck region is tapered in a direction away from the receiving region, and the neck region defines the second opening arranged away from the first opening.

12. The bioparticle contactless processing apparatus according to claim 11, wherein the triggering device includes:
two electrode pads located in the receiving region and respectively disposed on inner walls of the two working segments; and
a power source electrically coupled to the two electrode pads, wherein, when the target bioparticle is captured to be located in the receiving region, the power source is configured to drive the two electrode pads to apply an electric field to the target bioparticle, whereby an electroporation is formed in the target bioparticle for enabling the outer cell layer to have the predetermined permeability.

13. The bioparticle contactless processing apparatus according to claim 11, wherein the triggering device includes:
two electrode pads located in the neck region and respectively disposed on inner walls of the two working segments; and
a power source electrically coupled to the two electrode pads, wherein, when the target bioparticle is captured to be located in the neck region, the power source is configured to drive the two electrode pads to apply an electric field to the target bioparticle, whereby an electroporation is formed in the target bioparticle for enabling the outer cell layer to have the predetermined permeability.

14. The bioparticle contactless processing apparatus according to claim 10, wherein the frame includes two channel segments respectively connected to one of the two opposite ends of the two working segments that defines the second opening, and wherein the two channel segments define a third opening that is arranged away from the second opening and that is greater than the second opening.

15. A bioparticle contactless processing apparatus, comprising:
an accommodating device for receiving a liquid specimen that has a plurality of bioparticles, wherein the accommodating device includes:
a light sensing structure including a first substrate, a first electrode layer formed on the first substrate, and a photoelectric layer that is formed on the first substrate;
a mating structure spaced apart from the light sensing structure, wherein at least one of the mating structure and the light sensing structure is transparent, and the mating structure includes a second substrate and a second electrode layer that is formed on the second substrate and that faces toward the light sensing structure; and
a frame arranged between the light sensing structure and the mating structure, wherein the frame has two working segments facing toward each other and having two opposite ends that respectively define a first opening and a second opening being smaller than the first opening, and wherein at least one of the bioparticles is defined as a target bioparticle having a particle size being greater than the second opening, and an outer cell layer of the target bioparticle has an original permeability; and
a triggering device disposed corresponding in position to the frame, wherein, when the target bioparticle is located between the two working segments by passing through the first opening, the triggering device is configured to trigger the outer cell layer of the target bioparticle, whereby the outer cell layer has a predetermined permeability being greater than the original permeability.
